(19) United States
(12) Patent Application Publication
(10) Pub. No.: US 2010/0119646 A1
(43) Pub. Date: May 13, 2010

(54) Dihydroeugenol as Additive for feed

(75) Inventors: Daniel D'Orazio, Halten (CH); Antoine Saizieu-De, Brunstatt (FR); Goede Schueler, Weil am Rhein (DE); Aurelia Seon, Mulhouse (FR); Carlos Simoes-Nunes, Village-Neuf (FR)

Correspondence Address: NIXON & VANDERHYE, PC, 901 NORTH GLEBE ROAD, 11TH FLOOR, ARLINGTON, VA, 22203, US

(73) Assignee: DSM IP ASSETS B.V., HEERLEN (NL)

(21) Appl. No.: 12/525,614

(22) PCT Filed: Jan. 31, 2008
(86) PCT No.: PCT/EP08/00748

§ 371 Date: Aug. 3, 2009

(30) Foreign Application Priority Data
Feb. 2, 2007 (EP) 07002259.5

(51) Int. Cl. A23K 1/18; C07C 41/00; A23D 9/013; A23L 1/30

(52) U.S. Cl. 426/2; 568/653; 426/531; 426/72

(57) ABSTRACT

The present invention relates to the use of a selected group of phenol derivatives as components of animal feed or feed additives for the improvement of animal performance as well as to the corresponding animal feed or feed additives containing them.

The present invention relates to a selected group of phenol derivatives as components of animal feed or feed additives, as well as to compositions, feed additives and feed containing them.

The term feed or feed composition means any compound, preparation, mixture, or composition suitable for, or intended for intake by an animal.

More particular, the present invention relates to a nutraceutical composition for animals comprising as active ingredient dihydroeugenol (2-methoxy-4-propylphenol), and/or coniferyl alcohol and/or methylguiacol and/or isoeugenol and derivatives or metabolites thereof.

The term “nutraceutical” as used herein denotes a usefulness in both the nutritional and pharmaceutical field of application. Thus, the nutraceutical compositions can find use as a complete animal feed (diet), as supplement to animal feed, and as pharmaceutical formulations for enteral or parenteral application which may be solid formulations, or liquid formulations.

The term animal includes all animals including human. Examples of animals are non-ruminants, and ruminants. Ruminant animals include, for example, animals such as sheep, goat, and cattle, e.g. cow such as beef cattle and dairy cows. In a particular embodiment, the animal is a non-ruminant animal. Non-ruminant animals include pet animals, e.g. horses, cats and dogs; mono-gastric animals, e.g. pig or swine (including, but not limited to, piglets, growing pigs, and sows); poultry such as turkeys, ducks and chickens (including but not limited to broiler chicks, layers); fish (including but not limited to salmon, trout, tilapia, catfish and carp); and crustaceans (including but not limited to shrimp and prawn).

For example Dihydroeugenol is known as naturally occurring compound derived from plants which exhibits antimicrobial activities. EP 1 238 650 A1 discloses an antimicrobial flavor and oral care composition containing for example dihydroeugenol. The novel composition are said to be especially used in toothpastes, mouthwashes or food for inhibiting oral pathogenic microorganisms, and preventing dental caries, paradentosis or halitosis in human. As advantages it is pointed out that the antimicrobial flavors and flavor compositions have little influential on intestinal bacteria.

The present inventors now surprisingly found that the compounds specified herein above have a great potential for use in animal feed, e.g. for improving the feed conversion ratio (FCR) and/or for modulation of the gut flora.

Therefore, the present invention provides the use of a selected group of phenol derivatives as components of animal feed or feed additives wherein these compounds are defined by the formula (I)

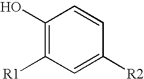

and wherein
R1 is hydrogen or hydroxyl or OR3, wherein the residues R3 is a lower alkyl or lower alkenyl residue;
R2 is hydrogen or a lower alkyl or lower alkoxy or lower alkenyl residue.

The invention further provides the use of these derivatives for the preparation of compositions improving the performance of animals, especially having activity as modulators of the gastrointestinal microflora and which are applicable via animal feed.

The preferred compounds of formula (I) are 2 dihydroeugenol (2-methoxy-4-propylphenol), coniferyl alcohol, methylguiacol and isoeugenol; most preferred is the compound dihydroeugenol (2-methoxy-4-propylphenol according to formula II

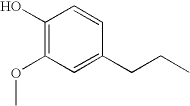

Finally, the present invention provides animal feed additives on the basis of a compound defined above, a derivative or metabolite thereof and animal feed containing as an additive such a compound, a derivative or metabolite thereof.

The compounds of formula (I) are either commercially available or can easily be prepared by a skilled person using processes and methods well-known in the prior art. In particular, dihydroeugenol can be isolated and purified by methods known per se, e.g. by adding a solvent such as methanol to induce the separation of the crude product from a plant extract, chromatography and/or crystallization of the collected crude product. Dihydroeugenol can also be synthesized from eugenol, for example as follows: 100 mg eugenol may be dissolved in EtOH and hydrogenated on 10% Pd—C at room temperature for 5 h. After filtration the reaction mixture can be evaporated to dryness. Finally the residue may be distilled by steam distillation to give dihydroeugenol as a yellow oil.

Compounds according to the present invention and compositions containing them improve the performance of animals, viz. their general health status and during breeding their weight gain. The derivatives of the present invention can especially be regarded as modulators of the gastrointestinal microflora of the animals which is of importance for their health status including weight gain. Positive effects with this respect of the said compounds may be based at least partially, on their inhibitory effects on potentially pathogenic microorganisms, e.g. on antibacterial activity. Therefore, they can be used as feed additives or for the preparation thereof and of feed by mixing or processing them with conventional animal feed or components thereof for all kinds of animals in amounts to provide the required or desired daily uptake. Preferred animals which may be in need of such additives comprise mammals, e.g. ruminants, pigs, calves, horses, pets, birds, e.g. poultry (chickens, hens, geese, ducks, turkeys), fish and zoo animals. A group of animals for the breeding of which the present acyl derivatives are preferably useful are stock animals.

The phenol derivatives according to the invention may be administrated to the animals as a component of a nutraceutical composition which is conventionally fed to animals. Thus, the compounds may be suitably administered to the animals as a component of the animal feed or in their drinking water. The compounds may also be administrated to the animals as a component of a pharmaceutical composition.

The normal daily dosage of a compound of formula I provided to an animal by feed intake depends upon the kind of animal and its condition. Normally this dosage should be in the range of from about 50 to about 1000 mg, preferably from about 100 to about 500 mg compound per kg of feed.

In a preferred embodiment of the invention dihydroeugenol or a derivative of dihydroeugenol being used in an amount sufficient to provide a daily dosage of 2.5 mg per kg body weight to about 50 mg per kg body weight of the subject to which it is to be administered.

Dihydroeugenol or a derivative thereof may be used in combination with conventional ingredients present in an animal feed composition (diet) such as calcium carbonates, electrolytes such as ammonium chloride, proteins such as soya bean meal, wheat, starch, sunflower meal, corn, meat and bone meal, amino acids, animal fat, vitamins and trace minerals.

In a particular embodiment, the invention relates to methods for using dihydroeugenol in animal feed for improving the Feed Conversion Ratio (FCR) and/or for modulation of the gut microflora. In alternative embodiments, dihydroeugenol improves animal feed digestibility, and/or maintains animal health by aiding in proper digestion and/or supporting immune system function.

The FCR may be determined on the basis of a broiler chicken growth trial comprising a first treatment in which dihydroeugenol is added to the animal feed in a suitable concentration per kg feed, and a second treatment (control) with no addition of dihydroeugenol to the animal feed.

As it is generally known, an improved FCR is lower than the control FCR. In particular embodiments, the FCR is improved (i.e., reduced) as compared to the control by at least 1.0%, preferably at least 1.5%, 1.6%, 1.7%, 1.8%, 1.9%, 2.0%, 2.1%, 2.2%, 2.3%, 2.4%, or at least 2.5%.

The term “gut” as used herein designates the gastrointestinal or digestive tract (also referred to as the alimentary canal) and it refers to the system of organs within multicellular animals which takes in food, digests it to extract energy and nutrients, and expels the remaining waste.

The term gut “microflora” as used herein refers to the natural microbial cultures residing in the gut and maintaining health by aiding in proper digestion and/or supporting immune system function.

The term “modulate” as used herein in connection with the gut microflora generally means to change, manipulate, alter, or adjust the function or status thereof in a healthy and normally functioning animal, i.e. a non-therapeutic use.

The following are non-limiting particular examples of the gut microflora modulation effect obtained by dihydroeugenol and derivatives thereof:

(i) a decrease in the number of Escherichia coli in vivo, for example in piglets and/or broilers, preferably determined after cultivation of ileo-rectal and/or caecal contents, respectively, on Coli-ID chromogenic media, aerobically, at 37° C. for 24 hours;
(ii) a decrease in the number of other Enterobacteriaceae (other than E. coli) in vivo, for example in piglets, preferably determined after cultivation of ileo-rectal contents on a Coli-ID chromogenic media, aerobically, at 37° C. for 24 hours;
(iii) a decrease in the number of Enterococcus spp. in vivo, for example in piglets, preferably determined after cultivation of ileo-rectal contents on an Enterococci agar, aerobically, at 44° C. for 48 hours; and/or

Still further, also in relation to the gut microflora modulating effect, and with reference to a control without dihydroeugenol of the invention, dihydroeugenol of the invention preferably:

(iv) does substantially influence, e.g. reduce, the growth in vitro of harmful micro-organisms, such as bacteria, for example as isolated from piglet and/or broiler intestinal contents.

Particular examples of compositions of the invention are the following:
An animal feed additive comprising (a) dihydroeugenol, or a derivative thereof (b) at least one fat-soluble vitamin, (c) at least one water-soluble vitamin, (d) at least one trace mineral, and/or (e) at least one macro mineral;
An animal feed composition comprising dihydroeugenol or a derivative thereof and a crude protein content of 50 to 800 g/kg feed.

The so-called premixes are examples of animal feed additives of the invention. A premix designates a preferably uniform mixture of one or more micro-ingredients with diluent and/or carrier. Premixes are used to facilitate uniform dispersion of micro-ingredients in a larger mix.

For the use in animal feed, however, dihydroeugenol need not be that pure; it may e.g. include other compounds and derivatives.

In the present context, the term Feed Conversion Ratio, or FCR, is used synonymously with the term feed conversion. The FCR is calculated as the feed intake in g/animal relative to the weight gain in g/animal.

Further, optional, feed-additive ingredients are coloring agents, e.g. carotenoids such as beta-carotene, astaxanthin, and lutein; aroma compounds; stabilisers; antimicrobial peptides; polyunsaturated fatty acids; reactive oxygen generating species; and/or at least one enzyme selected from amongst phytase (EC 3.1.3.8 or 3.1.3.26); xylanase (EC 3.2.1.8); galactanase (EC 3.2.1.89); alpha-galactosidase (EC 3.2.1.22); protease (EC 3.4.), phospholipase A1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Usually fat- and water-soluble vitamins, as well as trace minerals form part of a so-called premix intended for addition to the feed, whereas macro minerals are usually separately added to the feed. Either of these composition types, when enriched with dihydroeugenol is an animal feed additive of the invention.

The following are non-exclusive lists of examples of these components:

Examples of fat-soluble vitamins are vitamin A, vitamin D3, vitamin E, and vitamin K, e.g. vitamin K3.

Examples of water-soluble vitamins are vitamin B12, biotin and choline, vitamin B1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

Animal feed compositions or diets have a relatively high content of protein. Poultry and pig diets can be characterized as indicated in Table B of WO 01/58275, columns 2-3. Fish diets can be characterized as indicated in column 4 of this Table B. Furthermore such fish diets usually have a crude fat content of 200-310 g/kg.

WO 01/58275 corresponds to U.S. Ser. No. 09/779,334 which is hereby incorporated by reference.

An animal feed composition according to the invention has a crude protein content of 50-800 g/kg, and furthermore comprises at least dihydroeugenol and/or at least a derivative thereof as described and/or claimed herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen by, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein or protein source. It may also contain animal protein, such as Meat and Bone Meal, and/or Fish Meal, typically in an amount of 0-25%. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Vegetable proteins may be derived from vegetable protein sources, such as legumes and cereals, for example materials from plants of the families Fabaceae (Leguminosae), Cruciferaceae, Chenopodiaceae, and Poaceae, such as soy bean meal, lupin meal and rapeseed meal.

In a particular embodiment, the vegetable protein source is material from one or more plants of the family Fabaceae, e.g. soybean, lupine, pea, or bean.

In another particular embodiment, the vegetable protein source is material from one or more plants of the family Chenopodiaceae, e.g. beet, sugar beet, spinach or quinoa.

Other examples of vegetable protein sources are rapeseed, sunflower seed, cotton seed, and cabbage.

Other examples of vegetable protein sources are cereals such as barley, wheat, rye, oat, maize (corn), rice, triticale, and sorghum.

In still further particular embodiments, the animal feed composition of the invention contains 0-80% maize; and/or 0-80% sorghum; and/or 0-70% wheat; and/or 0-70% Barley; and/or 0-30% oats; and/or 0-30% rye; and/or 0-40% soybean meal; and/or 0-25% fish meal; and/or 0-25% meat and bone meal; and/or 0-20% whey.

Animal diets can e.g. be manufactured as mash feed (non pelleted) or pelleted feed.

Typically, the milled feed-stuffs are mixed and sufficient amounts of essential vitamins and minerals are added according to the specifications for the species in question. Dihydroeugenol or the derivative thereof can be added as solid or liquid formulations.

The final dihydroeugenol concentration in the diet is within the range of 50-10000 mg per kg diet, for example in the range of 200-1000 mg per kg animal diet.

Dihydroeugenol or the derivative thereof should of course be applied in an effective amount, i.e. in an amount adequate for improving feed conversion.

It is at present contemplated that dihydroeugenol is administered in one or more of the following amounts (dosage ranges): 0.01-500; 0.01-200; 0.01-100; 0.5-100; 1-50; 5-100; 10-100; 0.05-50; 1-10; or 0.10-10, all these ranges being in mg dihydroeugenol per kg feed (ppm).

Other compounds which may be used as feed additives and which show similar effects as dihydroeugenol are 4-cinnamylphenol, 2-cinnamylphenol, 2-cinnamyl-4-methyl-phenol, 2-cinnamyl-4-methoxy-phenol, 2,6-Dimethylphenol, 2,5-Dimethylphenol, 4-methylphenol (p-cresol), 2-methylphenol, aureusidin, derivatives, metabolites or analogues thereof.

The following examples further illustrate the invention, but they should not be construed as limiting the invention.

### EXAMPLE 1

### Animal Feed Additive

An animal feed additive is prepared by adding 20 g of dihydroeugenol to the following premix (per kilo of premix):

| | | |
|---|---|---|
| 1100000 | IE | Vitamin A |
| 300000 | IE | Vitamin D3 |
| 4000 | IE | Vitamin E |
| 250 | mg | Vitamin B1 |
| 800 | mg | Vitamin B2 |
| 1200 | mg | Ca-D-Panthothenate |
| 500 | mg | Vitamin B6 |
| 2.5 | mg | Vitamin B12 |
| 5000 | mg | Niacin |
| 10000 | mg | Vitamin C |
| 300 | mg | Vitamin K3 |
| 15 | mg | Biotin |
| 150 | mg | Folic acid |
| 50004 | mg | Cholin chloride |
| 6000 | mg | Fe |
| 3000 | mg | Cu |
| 5400 | mg | Zn |
| 8000 | mg | Mn |
| 124 | mg | I |
| 60 | mg | Co |
| 29.7 | mg | Se |
| 9000 | mg | Lasalocid Sodium (Avatec) |

| | |
|---|---|
| 17.3% | Ca |
| 0.8% | Mg |
| 11.7% | Na |

### EXAMPLE 2

### Animal Feed

A broiler grower diet having the following composition (%, w/w) is prepared by mixing the ingredients. Wheat, rye and SBM 48 are available from Moulin Moderne Hirsinque, Hirsingue, France. After mixing, the feed is pelleted at a desired temperature, e.g. about 70° C. (3×25 mm).

| | |
|---|---|
| Wheat | 46.00 |
| Rye | 15.00 |
| Soy Bean Meal (SBM 48) | 30.73 |
| Soybean oil | 4.90 |
| DL-Methionine | 0.04 |
| DCP (Di-Calcium Phosphate) | 1.65 |
| Limestone | 0.43 |
| Salt | 0.15 |
| TiO2 | 0.10 |
| Animal feed additive (above) | 1.00 |

The resulting animal feed comprises 200 mg dihydroeugenol per kg (200 ppm).

### EXAMPLE 3

A piglet food containing dihydroeugenol can be prepared by mixing the following ingredients together using a conventional mixing apparatus at room temperature.

| Ingredient | Amount (kg) |
|---|---|
| Wheat | 32.6 |
| Maize | 18.7 |
| Rice | 5.0 |
| Wheat bran | 9.0 |
| Soybean meal | 23.0 |
| Soy oil | 2.0 |
| Wheat starch | 4.5 |
| Minerals * | 2.9 |
| Synthetic amino acids premix ** | 0.8 |
| Vitamins and trace elements premix *** | 1.0 |
| Dihydroeugenol premix (10% in wheat starch) | 0.5 |

In principle the dihydroeugenol premix may contain 1-20% of the dihydroeugenol.
* Sea salt, dicalcium phosphate and calcium carbonate;
** Lysine, methionine and threonine;
*** Vitamins A, E, D3, K3, B1, B2, B6, B12, C, biotine, folic acid, niacin, pantothenic acid, choline chloride, copper sulphate, iron sulphate, manganese oxide, zinc oxide, cobalt carbonate, calcium iodide and sodium selenite.

### EXAMPLE 4

A growing pig food containing dihydroeugenol can be prepared by mixing the following ingredients together using a conventional mixing apparatus at room temperature.

| Ingredient | Amount (kg) |
|---|---|
| Soybean meal | 18.0 |
| Maize | 52.3 |
| Barley | 14.0 |
| Oat meal | 6.0 |
| Wheat bran | 5.2 |
| Soy oil | 2.0 |
| Minerals * | 1.5 |
| Synthetic amino acids premix ** | 0.5 |
| Vitamins and trace elements premix *** | 1.0 |
| Dihydroeugenol premix (10% in wheat starch) | 0.5 |

In principle the dihydroeugenol premix may contain 1-20% of the dihydroeugenol derivative.

### EXAMPLE 5

A broiler chicken food (“starter”) containing dihydroeugenol can be prepared by mixing the following ingredients together using a conventional mixing apparatus at room temperature.

| Ingredient | Amount (kg) |
|---|---|
| Soybean meal | 34.50 |
| Maize | 20.00 |
| Wheat | 37.80 |
| Soy oil | 3.13 |
| Minerals * | 2.90 |
| Synthetic amino acids premix ** | 0.17 |
| Vitamins and trace elements premix *** | 1.00 |
| Dihydroeugenol premix (10% in wheat starch) | 0.50 |

In principle the dihydroeugenol premix may contain 1-20% of the dihydroeugenol derivative.

### EXAMPLE 6

A broiler chicken food (“grower”) containing dihydroeugenol can be prepared by mixing the following ingredients together using a conventional mixing apparatus at room temperature.

| Ingredients | Amount (kg) |
|---|---|
| Soybean meal | 31.2 |
| Maize | 20.0 |
| Wheat | 41.3 |
| Soy oil | 3.4 |
| Minerals * | 2.5 |
| Synthetic amino acids premix ** | 0.1 |
| Vitamins and trace elements premix *** | 1.0 |
| Dihydroeugenol premix (10% in wheat starch) | 0.5 |

In principle the dihydroeugenol premix may contain 1-20% of the dihydroeugenol derivative.

### EXAMPLE 7

### Evaluation of the Antimicrobial Activity of Dihydroeugenol

Starting from a glycerol stock, a pre-culture of E. coli 0.96 was performed in 10 ml Tryptic Soy Broth (TSB, Merck) at 37° C., shaking at 250 rpm over-night.

This preculture was diluted in TSB to get a bacterial suspension with approximately 4×10⁴ cfu/ml. 2-methoxy-4-propylphenol was dissolved in DMSO. 5 μl of each dilution were distributed into a 96 well plate in triplicates and further diluted with 45 μl TSB. Controls consisting of Spectinomycin (1 mg/ml final concentration) and DMSO (2.5% final) were also included. Finally, 150 μl of the bacterial suspension were added to each well.

At time zero the OD_{595 nm} was measured to take into account any turbidity due to precipitated compounds. The plates were then incubated over night at 37° C. in a humid atmosphere and shaken at 200 rpm.

After 24 hours the OD_{595 nm} was measured to calculate the percent inhibition.

The percent inhibition was calculated as follows: ((HC−OD)/(HC−LC))*100 where HC is the average of the OD_{595 nm} measured with 2.5% DMSO, LC is the average of the OD_{595 nm} measured with 1 mg/ml spectinomycine and OD is the OD_{595 nm} measured for a single well containing the test compound.

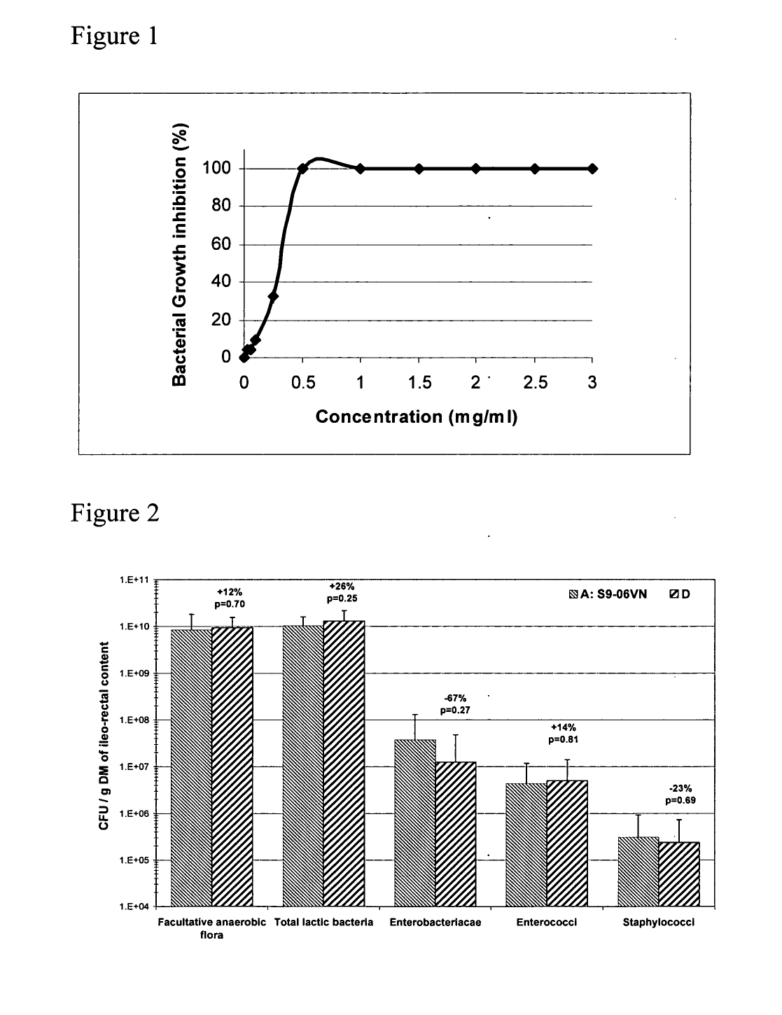

The results are shown in FIG. 1.

### EXAMPLE 8

### Gut Microflora Modulation In Vivo

The influence of 2-methoxy-4-propylphenol on the gut microflora was evaluated in vivo in piglets.

This study, which was performed according to the French legal regulations on experiments with live animals, was performed to evaluate the effect of 300 ppm of 2-methoxy-4-propylphenol (300 mg purified 2-methoxy-4-propylphenol per kg of feed) on piglet gut microflora.

Ten piglets (hybrids of Large-White, Landrace, and Piétrain, obtained from GAEC Leclerc, Ostheim, France) of an initial body weight of 20.1±1.3 kg were submitted to an ileo-rectal anastomosis (connecting the terminal ileum to the end of the rectum, bypassing the caecum and the colon). In such pigs, the microflora of the terminal ileum can be collected at the anus level and is representative of the bacterial population of all the consecutive digestive parts of the intestines. After surgery, during recovery from surgery, and during the experimental period the animals were placed in metabolic cages allowing an easy sampling of ileo-rectal contents.

During the experimental period of six weeks, the piglets were fed each and alternatively (in a double-latin-square design, to reduce the effect of individual variation and also any potential influence of the sequence of the treatments) a basal diet supplemented or not with test compounds. The diets were composed as follows:

Diet A: KLIBA, available from Provimi-Kliba, Kaiseraugst, Switzerland, with 18% soybean meal, 53% maize, 13% barley, 6% oat meal, 5.4% wheat bran, 1% soy oil, 3.6% minerals, vitamins and synthetic amino acids (w/w).

Diet D: Diet A with the addition of 300 mg/kg of 2-methoxy-4-propylphenol

Diets B, C and E included other test compounds of no relevance for the present invention.

The test compounds were incorporated into the diets in a Buhler mixer (Buhler, Aschwill, Switzerland). The experimental diets were prepared and administered to the animals in a mash form.

The experimental diets were allowed to the animals at the level of 2 kg per day distributed in two equal meals at 8:00 and 15:30.

Ileo-rectal contents were sampled from each animal at the two last days of each treatment period, and the concentrations of dry matter and of the different constituents of the microflora were determined.

The animals did not show any symptoms of toxicosis or of illness during the experiment. Their daily weight gain during the observation was 0.3+/−0.05 kg. At the end of the experiment the animals were euthanized by lethal injection after tranquilisation.

Analysis of the microflora was performed as follows:

The dry matter contents of the samples, were determined after drying overnight at 105° C., 1 g of sample, according to the standard Association of Official Analytical Chemists (AOAC) procedure (1009) (Association of Official Analytical Chemists. (1990). (Official methods of analysis. 15th edition. Association of Official Analytical Chemists. Arlington.)

Immediately after emission, 10 g of ileo-rectal contents were transferred and homogenized into flasks containing 100 ml of reduced physiological salt solution (Merck, Darmstadt, Germany, catalogue n° 10582). Less than 5 min passed between emissions and removal of the ileal contents which were rapidly transferred to an anaerobic chamber (AES Cheminex, Combourg, France). Subsequently, the samples were serially diluted in 10-fold steps using physiological salt solution from 10-1 to 10-8 (wt/vol). All bacterial counts were achieved with two replicate plates.

Total facultative anaerobic counts represent the average number of colonies that grew on Brucella agar (Merck, Darmstadt, Germany, catalogue n° 1.10490) supplemented with sheep blood (5% vol/vol), hemine (10 mg/ml) and vitamin K1 (10 mg/ml). Plates were incubated in an anaerobic cabinet at 37° C. during 5 days.

Lactic acid bacteria were enumerated on MRS agar (Merck, Darmstadt, Germany, catalogue n° 110660). Plates were incubated in an anaerobic cabinet at 37° C. during 48 h.

Enterobacteriacae were counted on V.R.B.D agar (Merck, Darmstadt, Germany, catalogue n° 110275) after being incubated aerobically at 37° C. during 24 h.

Enterococcus spp were evaluated on Enterococci agar (Merck, Darmstadt, Germany, catalogue no 65009) and Staphylococcus spp on Baird Parker agar (AES Cheminex, Combourg, France, catalogue n° AEB150302) after aerobic incubation at 37° C. during 48 h. After heating the sample in a water bath at 80° C. during 10 min, Clostridium perfringens was isolated using TSN agar (BioMérieux, Marcy l'Etoile, France, catalogue n° 51048) incubated in an anaerobic chamber at 46° C. during 24 hours.

Identities of representative colonies were furthermore confirmed by microscopic examination after gram staining and biochemical testing using the appropriate API system (BioMérieux, Marcy l'Etoile, France).

The respective bacterial counts (number of colony forming units (CFU) per gram of ileal content dry matter (DM)) are presented in FIG. 2 (FIG. 2: Ileo-rectal microflora in ten piglets submitted to an ileo-rectal anastomosis fed a diet without or with supplementation of 300 ppm of 2-methoxy-4-propylphenol), which also shows the amendment in the respective bacterial counts caused by addition of 300 ppm of 2-methoxy-4-propylphenol, relative to the control.

In this assay, no significant differences in the bacterial counts were found, only numeric effects were observed, but some times they were very strong.

A neutral effect of 2-methoxy-4-propylphenol was seen on the mean of total facultative anaerobic bacteria and on the total lactic acid bacteria, which are representatives of the beneficial bacteria of the intestinal microbiota. In both bacterial counts were increased respectively by 12% and 26% relative to the control.

A positive effect of 2-methoxy-4-propylphenol was seen on the mean of total Enterobacteriacae, where the counts were decreased by 67% relative to the control. The main component of the Enterobacteriaceae population in the piglet microflora, are Escherichia coli, which some might be potentially pathogenic.

A neutral effect of 2-methoxy-4-propylphenol was seen on the population of Enterococcus spp and Staphylococcus spp. Clostridium perfringens concentration found were too small to draw any conclusion.

### EXAMPLE 9

### Evaluation of Antimicrobial Activity and Gut Microflora Modulation of 4-cinnamylphenol

Starting from a glycerol stock, a pre-culture of E. coli 0.96 was performed in 10 ml Tryptic Soy Broth (TSB, Merck) at 37° C., shaking at 250 rpm over-night.

This preculture was diluted in TSB to get a bacterial suspension with approximately 4×10⁴ cfu/ml. 4-cinnamylphenol was dissolved in DMSO. 5 μl of each dilution were distributed into a 96 well plate in triplicates and further diluted with 45 μl TSB. Controls consisting of Spectinomycin (1 mg/ml final concentration) and DMSO (2.5% final) were also included. Finally, 150 μl of the bacterial suspension were added to each well.

At time zero the OD_{595 nm} was measured to take into account any turbidity due to precipitated compounds. The plates were then incubated over night at 37° C. in a humid atmosphere and shaken at 200 rpm.

After 24 hours the OD_{595 nm} was measured to calculate the percent inhibition.

The percent inhibition was calculated as follows: ((HC−OD)/(HC−LC))*100 where HC is the average of the OD_{595 nm} measured with 2.5% DMSO, LC is the average of the OD_{595 nm} measured with 1 mg/ml spectinomycine and OD is the OD_{595 nm} measured for a single well containing the test compound.

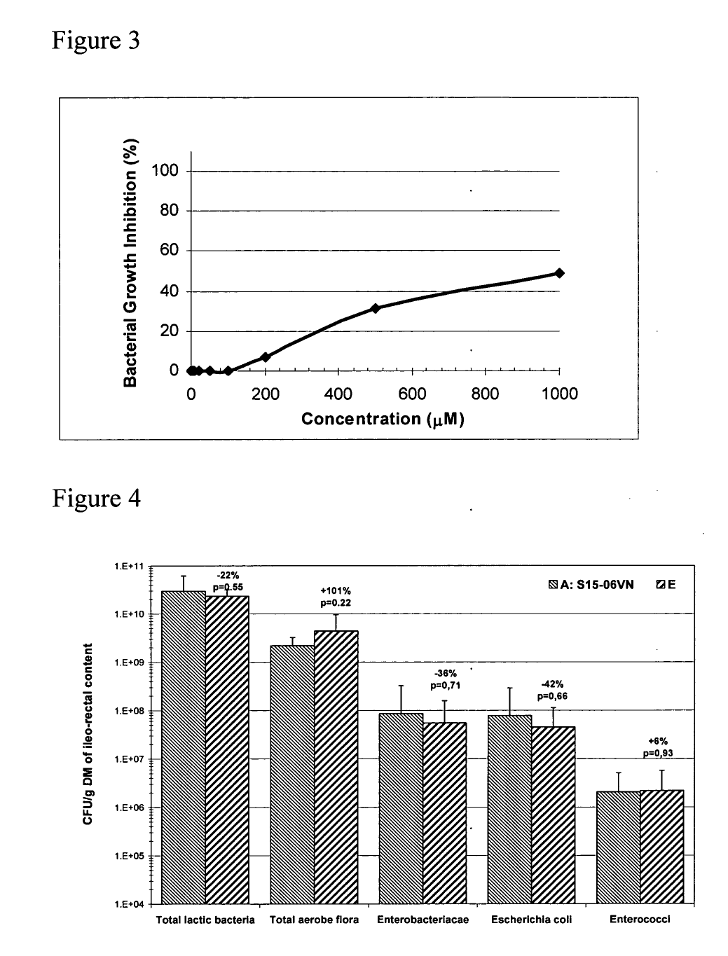

The results are shown in FIG. 3.

The influence of 4-cinnamylphenol on the gut microflora was evaluated in vivo in piglets.

This study, which was performed according to the French legal regulations on experiments with live animals, was performed to evaluate the effect of 300 ppm of 4-cinnamylphenol (300 mg purified 4-cinnamylphenol per kg of feed) on piglet gut microflora.

Ten piglets (hybrids of Large-White, Landrace, and Piétrain, obtained from GAEC Leclerc, Ostheim, France) of an initial body weight of 25.08±1.9 kg were submitted to an ileo-rectal anastomosis (connecting the terminal ileum to the end of the rectum, bypassing the caecum and the colon). In such pigs, the microflora of the terminal ileum can be collected at the anus level and is representative of the bacterial population of all the consecutive digestive parts of the intestines. After surgery, during recovery from surgery, and during the experimental period the animals were placed in metabolic cages allowing an easy sampling of ileo-rectal contents.

During the experimental period of six weeks, the piglets were fed each and alternatively (in a double-latin-square design, to reduce the effect of individual variation and also any potential influence of the sequence of the treatments) a basal diet supplemented or not with test compounds. The diets were composed as follows:

Diet A: KLIBA, available from Provimi-Kliba, Kaiseraugst, Switzerland, with 18% soybean meal, 53% maize, 13% barley, 6% oat meal, 5.4% wheat bran, 1% soy oil, 3.6% minerals, vitamins and synthetic amino acids (w/w).

Diet E: Diet A with the addition of 300 mg/kg of 4-cinnamylphenol

Diets B, C and D included other test compounds of no relevance for the present invention.

The test compounds were incorporated into the diets in a Buhler mixer (Buhler, Aschwill, Switzerland). The experimental diets were prepared and administered to the animals in a mash form.

The experimental diets were allowed to the animals at the level of 2 kg per day distributed in two equal meals at 8:00 and 15:30.

Ileo-rectal contents were sampled from each animal at the two last days of each treatment period, and the concentrations of dry matter and of the different constituents of the microflora were determined.

The animals did not show any symptoms of toxicosis or of illness during the experiment. Their daily weight gain during the observation was 0.152+/−0.119 kg. At the end of the experiment the animals were euthanized by lethal injection after tranquilisation.

Analysis of the microflora was performed as follows:

The dry matter contents of the samples, were determined after drying overnight at 105° C., 1 g of sample, according to the standard Association of Official Analytical Chemists (AOAC) procedure (1009) (Association of Official Analytical Chemists. (1990). (Official methods of analysis. 15th edition. Association of Official Analytical Chemists. Arlington.)

Immediately after emission, 10 g of ileo-rectal contents were transferred and homogenized into flasks containing 100 ml of reduced physiological salt solution (Merck, Darmstadt, Germany, catalogue n° 10582). Less than 5 min passed between emissions and removal of the ileal contents which were rapidly transferred to an anaerobic chamber (AES Cheminex, Combourg, France). Subsequently, the samples were serially diluted in 10-fold steps using physiological salt solution from 10-1 to 10-8 (wt/vol). All bacterial counts were achieved with two replicate plates.

Total facultative anaerobic counts represent the average number of colonies that grew on Brucella agar (Merck, Darmstadt, Germany, catalogue n° 1.10490) supplemented with sheep blood (5% vol/vol), hemine (10 mg/ml) and vitamin K1 (10 mg/ml). Plates were incubated in an anaerobic cabinet at 37° C. during 5 days.

Lactic acid bacteria were enumerated on MRS agar (Merck, Darmstadt, Germany, catalogue n° 110660). Plates were incubated in an anaerobic cabinet at 37° C. during 48 h.

Enterobacteriacae were counted on V.R.B.D agar (Merck, Darmstadt, Germany, catalogue n° 110275) after being incubated aerobically at 37° C. during 24 h.

Enterococcus spp were evaluated on Enterococci agar (Merck, Darmstadt, Germany, catalogue n° 65009) and Staphylococcus spp on Baird Parker agar (AES Cheminex, Combourg, France, catalogue n° AEB150302) after aerobic incubation at 37° C. during 48 h. After heating the sample in a water bath at 80° C. during 10 min, Clostridium perfringens was isolated using TSN agar (BioMérieux, Marcy l'Etoile, France, catalogue n° 51048) incubated in an anaerobic chamber at 46° C. during 24 hours.

Identities of representative colonies were furthermore confirmed by microscopic examination after gram staining and biochemical testing using the appropriate API system (BioMérieux, Marcy l'Etoile, France).

The respective bacterial counts (number of colony forming units (CFU) per gram of ileal content dry matter (DM)) are presented in FIG. 4 (Ileo-rectal microflora in ten piglets submitted to an ileo-rectal anastomosis fed a diet without or with supplementation of 300 ppm of 4-cinnamylphenol), which also shows the amendment in the respective bacterial counts caused by addition of 300 ppm of 2-methoxy-4-propylphenol, relative to the control.

In this assay, no significant differences in the bacterial counts were found, only numeric effects were observed, but some times they were very strong.

A negative effect of 4-cinnamylphenol was seen on the mean of total lactic acid bacteria where the counts were decreased by 22% relative to the control. But a positive effect of 4-cinnamylphenol was also seen on the mean of Enterobacteriacae and Escherichia coli where the counts were respectively decreased by 36% and 42% relative to the control.

Clostridium perfringens concentration found were too small to draw any conclusion.

### EXAMPLE 10

### Evaluation of Antimicrobial Activity and Gut Microflora Modulation of 2,6-dimethylphenol

Starting from a glycerol stock, a pre-culture of E. coli 0.96 was performed in 10 ml Tryptic Soy Broth (TSB, Merck) at 37° C., shaking at 250 rpm over-night.

This preculture was diluted in TSB to get a bacterial suspension with approximately 4×10⁴ cfu/ml. 2,6-dimethylphenol was dissolved in DMSO. 5 μl of each dilution were distributed into a 96 well plate in triplicates and further diluted with 45 μl TSB. Controls consisting of Spectinomycin (1 mg/ml final concentration) and DMSO (2.5% final) were also included. Finally, 150 μl of the bacterial suspension were added to each well.

At time zero the OD_{595 nm} was measured to take into account any turbidity due to precipitated compounds. The plates were then incubated over night at 37° C. in a humid atmosphere and shaken at 200 rpm.

After 24 hours the OD_{595 nm} was measured to calculate the percent inhibition.

The percent inhibition was calculated as follows: ((HC−OD)/(HC−LC))*100 where HC is the average of the OD_{595 nm} measured with 2.5% DMSO, LC is the average of the OD_{595 nm} measured with 1 mg/ml spectinomycine and OD is the OD_{595 nm} measured for a single well containing the test compound.

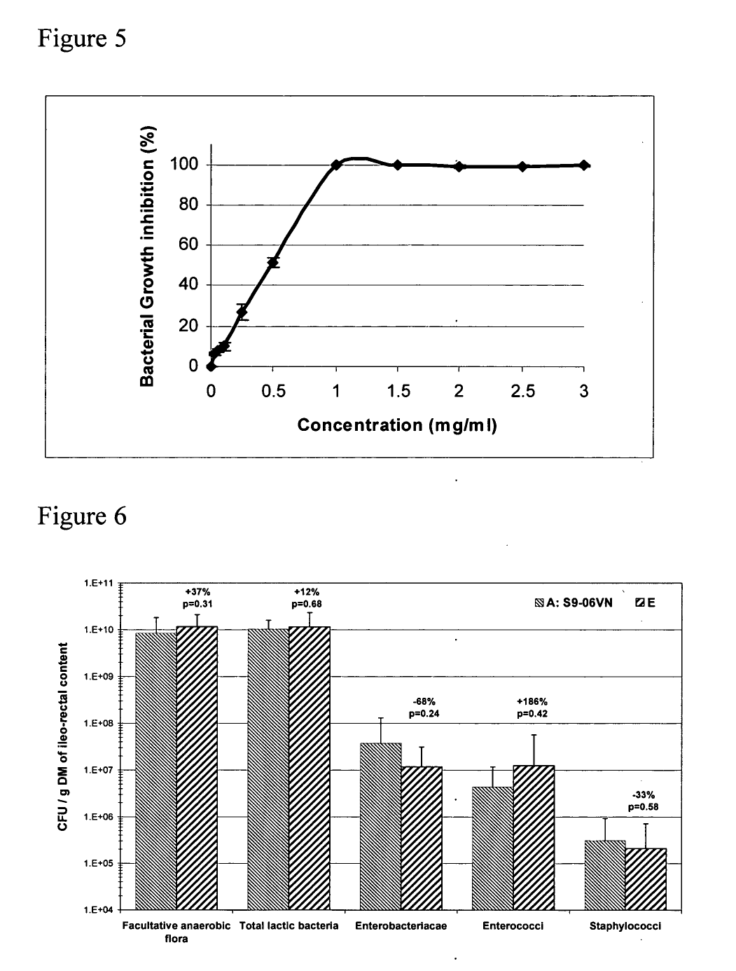

The results are shown in FIG. 5.

The influence of 2,6-dimethylphenol on the gut microflora was evaluated in vivo in piglets.

This study, which was performed according to the French legal regulations on experiments with live animals, was performed to evaluate the effect of 300 ppm of 2,6-dimethylphenol (300 mg purified 2,6-dimethylphenol per kg of feed) on piglet gut microflora.

Ten piglets (hybrids of Large-White, Landrace, and Piétrain, obtained from GAEC Leclerc, Ostheim, France) of an initial body weight of 20.1±1.3 kg were submitted to an ileo-rectal anastomosis (connecting the terminal ileum to the end of the rectum, bypassing the caecum and the colon). In such pigs, the microflora of the terminal ileum can be collected at the anus level and is representative of the bacterial population of all the consecutive digestive parts of the intestines. After surgery, during recovery from surgery, and during the experimental period the animals were placed in metabolic cages allowing an easy sampling of ileo-rectal contents.

During the experimental period of six weeks, the piglets were fed each and alternatively (in a double-latin-square design, to reduce the effect of individual variation and also any potential influence of the sequence of the treatments) a basal diet supplemented or not with test compounds. The diets were composed as follows:

Diet A: KLIBA, available from Provimi-Kliba, Kaiseraugst, Switzerland, with 18% soybean meal, 53% maize, 13% barley, 6% oat meal, 5.4% wheat bran, 1% soy oil, 3.6% minerals, vitamins and synthetic amino acids (w/w).

Diet E: Diet A with the addition of 300 mg/kg of 2,6-dimethylphenol.

Diets B, C and D included other test compounds of no relevance for the present invention.

The test compounds were incorporated into the diets in a Buhler mixer (Buhler, Aschwill, Switzerland). The experimental diets were prepared and administered to the animals in a mash form.

The experimental diets were allowed to the animals at the level of 2 kg per day distributed in two equal meals at 8:00 and 15:30.

Ileo-rectal contents were sampled from each animal at the two last days of each treatment period, and the concentrations of dry matter and of the different constituents of the microflora were determined.

The animals did not show any symptoms of toxicosis or of illness during the experiment. Their daily weight gain during the observation was 0.3+/−0.05 kg. At the end of the experiment the animals were euthanized by lethal injection after tranquilisation.

Analysis of the microflora was performed as follows:

The dry matter contents of the samples, were determined after drying overnight at 105° C., 1 g of sample, according to the standard Association of Official Analytical Chemists (AOAC) procedure (1009) (Association of Official Analytical Chemists. (1990). (Official methods of analysis. 15th edition. Association of Official Analytical Chemists. Arlington.)

Immediately after emission, 10 g of ileo-rectal contents were transferred and homogenized into flasks containing 100 ml of reduced physiological salt solution (Merck, Darmstadt, Germany, catalogue n° 10582). Less than 5 min passed between emissions and removal of the ileal contents which were rapidly transferred to an anaerobic chamber (AES Cheminex, Combourg, France). Subsequently, the samples were serially diluted in 10-fold steps using physiological salt solution from 10-1 to 10-8 (wt/vol). All bacterial counts were achieved with two replicate plates.

Total facultative anaerobic counts represent the average number of colonies that grew on Brucella agar (Merck, Darmstadt, Germany, catalogue n° 1.10490) supplemented with sheep blood (5% vol/vol), hemine (10 mg/ml) and vitamin K1 (10 mg/ml). Plates were incubated in an anaerobic cabinet at 37° C. during 5 days.

Lactic acid bacteria were enumerated on MRS agar (Merck, Darmstadt, Germany, catalogue n° 110660). Plates were incubated in an anaerobic cabinet at 37° C. during 48 h.

Enterobacteriacae were counted on V.R.B.D agar (Merck, Darmstadt, Germany, catalogue n° 110275) after being incubated aerobically at 37° C. during 24 h.

Enterococcus spp were evaluated on Enterococci agar (Merck, Darmstadt, Germany, catalogue n° 65009) and Staphylococcus spp on Baird Parker agar (AES Cheminex, Combourg, France, catalogue n° AEB150302) after aerobic incubation at 37° C. during 48 h. After heating the sample in a water bath at 80° C. during 10 min, Clostridium perfringens was isolated using TSN agar (BioMérieux, Marcy l'Etoile, France, catalogue n° 51048) incubated in an anaerobic chamber at 46° C. during 24 hours.

Identities of representative colonies were furthermore confirmed by microscopic examination after gram staining and biochemical testing using the appropriate API system (BioMérieux, Marcy l'Etoile, France).

The respective bacterial counts (number of colony forming units (CFU) per gram of ileal content dry matter (DM)) are presented in FIG. 6 (Ileo-rectal microflora in ten piglets submitted to an ileo-rectal anastomosis fed a diet without or with supplementation of 300 ppm of 2,6-dimethylphenol), which also shows the amendment in the respective bacterial counts caused by addition of 300 ppm of 2,6-dimethylphenol, relative to the control.

In this assay, no significant differences in the bacterial counts were found, only numeric effects were observed, but some times they were very strong.

A positive effect of 2,6-dimethylphenol was seen on the mean of total facultative anaerobic bacteria, where the counts were increased by 37% relative to the control. And a neutral effect of 2,6-dimethylphenol was seen on the total lactic acid bacteria which are representatives of the beneficial bacteria of the intestinal microbiota.

A positive effect of 2,6-dimethylphenol was seen on the mean of total Enterobacteriacae, where the counts were decreased by 68% relative to the control. The main component of the Enterobacteriaceae population in the piglet microflora, are Escherichia coli, which some might be potentially pathogenic.

A negative effect of 2,6-dimethylphenol was seen on the population of Enterococcus spp. Clostridium perfringens concentration found were too small to draw any conclusion.

### EXAMPLE 11

### Evaluation of Antimicrobial Activity and Gut Microflora Modulation of Aureusidin

Starting from a glycerol stock, a pre-culture of E. coli 0.96 was performed in 10 ml Tryptic Soy Broth (TSB, Merck) at 37° C., shaking at 250 rpm over-night.

This preculture was diluted in TSB to get a bacterial suspension with approximately 4×10⁴ cfu/ml. Aureusidin was dissolved in DMSO. 5 μl of each dilution were distributed into a 96 well plate in triplicates and further diluted with 45 μl TSB. Controls consisting of Spectinomycin (1 mg/ml final concentration) and DMSO (2.5% final) were also included. Finally, 150 μl of the bacterial suspension were added to each well.

At time zero the OD_{595 nm} was measured to take into account any turbidity due to precipitated compounds. The plates were then incubated over night at 37° C. in a humid atmosphere and shaken at 200 rpm.

After 24 hours the OD_{595 nm} was measured to calculate the percent inhibition.

The percent inhibition was calculated as follows: ((HC−OD)/(HC−LC))*100 where HC is the average of the OD_{595 nm} measured with 2.5% DMSO, LC is the average of the OD_{595 nm} measured with 1 mg/ml spectinomycine and OD is the OD_{595 nm} measured for a single well containing the test compound.

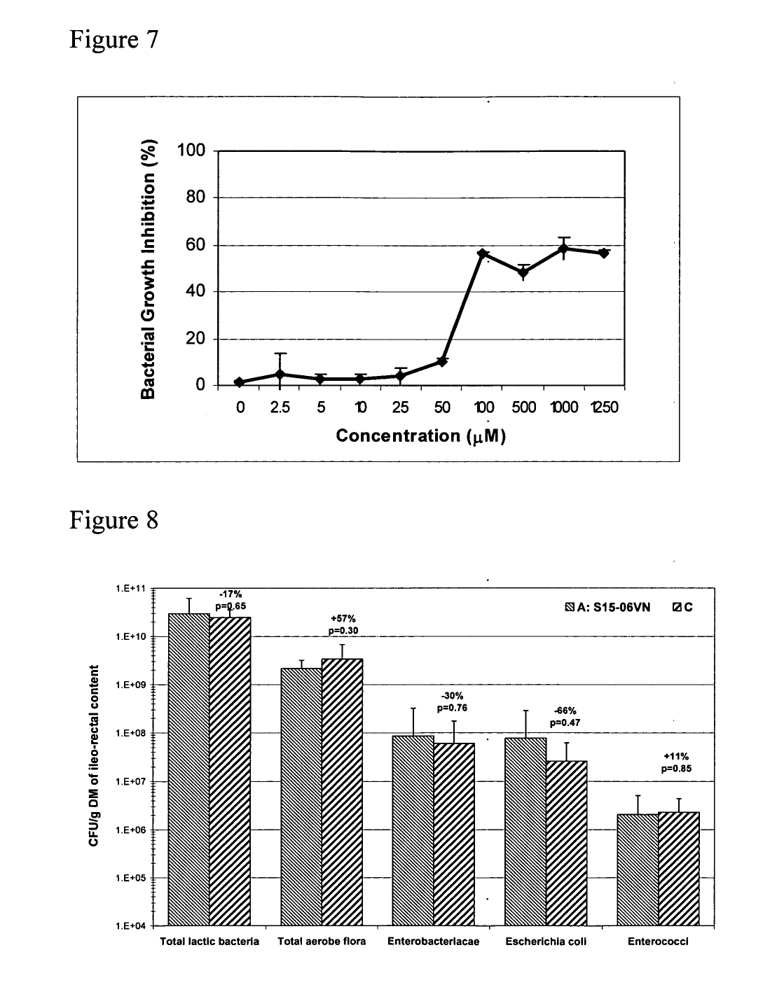

The results are shown in FIG. 7.

The influence of aureusidin on the gut microflora was evaluated in vivo in piglets.

This study, which was performed according to the French legal regulations on experiments with live animals, was performed to evaluate the effect of aureusidin on piglet gut microflora.

Ten piglets (hybrids of Large-White, Landrace, and Piétrain, obtained from GAEC Leclerc, Ostheim, France) of an initial body weight of 25.2±2.1 kg were submitted to an ileo-rectal anastomosis (connecting the terminal ileum to the end of the rectum, bypassing the caecum and the colon). In such pigs, the microflora of the terminal ileum can be collected at the anus level and is representative of the bacterial population of all the consecutive digestive parts of the intestines. After surgery, during recovery from surgery, and during the experimental period the animals were placed in metabolic cages allowing an easy sampling of ileo-rectal contents.

During the experimental period of six weeks, the piglets were fed each and alternatively (in a double-latin-square design, to reduce the effect of individual variation and also any potential influence of the sequence of the treatments) a basal diet supplemented or not with test compounds. The diets were composed as follows:

Diet A: KLIBA, available from Provimi-Kliba, Kaiseraugst, Switzerland, with 18% soybean meal, 53% maize, 13% barley, 6% oat meal, 5.4% wheat bran, 1% soy oil, 3.6% minerals, vitamins and synthetic amino acids (w/w).

Diet C: Diet A with the addition of 150 mg/kg of aureusidin

Diets B, D and E included other test compounds of no relevance for the present invention.

The test compounds were incorporated into the diets in a Buhler mixer (Buhler, Aschwill, Switzerland). The experimental diets were prepared and administered to the animals in a mash form.

The experimental diets were allowed to the animals at the level of 2 kg per day distributed in two equal meals at 8:00 and 15:30.

Ileo-rectal contents were sampled from each animal at the two last days of each treatment period, and the concentrations of dry matter and of the different constituents of the microflora were determined.

The animals did not show any symptoms of toxicosis or of illness during the experiment. Their daily weight gain during the observation was 0.3+/−0.05 kg. At the end of the experiment the animals were euthanized by lethal injection after tranquilisation.

Analysis of the microflora was performed as follows:

The dry matter contents of the samples, were determined after drying overnight at 105° C., 1 g of sample, according to the standard Association of Official Analytical Chemists (AOAC) procedure (1009) (Association of Official Analytical Chemists. (1990). (Official methods of analysis. 15th edition. Association of Official Analytical Chemists. Arlington.)

Immediately after emission, 10 g of ileo-rectal contents were transferred and homogenized into flasks containing 100 ml of reduced physiological salt solution (Merck, Darmstadt, Germany, catalogue n° 10582). Less than 5 min passed between emissions and removal of the ileal contents which were rapidly transferred to an anaerobic chamber (AES Cheminex, Combourg, France). Subsequently, the samples were serially diluted in 10-fold steps using physiological salt solution from 10-1 to 10-8 (wt/vol). All bacterial counts were achieved with two replicate plates.

Total facultative anaerobic counts represent the average number of colonies that grew on Brucella agar (Merck, Darmstadt, Germany, catalogue n° 1.10490) supplemented with sheep blood (5% vol/vol), hemine (10 mg/ml) and vitamin K1 (10 mg/ml). Plates were incubated in an anaerobic cabinet at 37° C. during 5 days.

Lactic acid bacteria were enumerated on MRS agar (Merck, Darmstadt, Germany, catalogue n° 110660). Plates were incubated in an anaerobic cabinet at 37° C. during 48 h.

Enterobacteriacae were counted on V.R.B.D agar (Merck, Darmstadt, Germany, catalogue n° 110275). Escherichia coli and other Enterobacteriacae were analysed on a Coli-ID chromogenic media (BioMérieux, Marcy l'Etoile, France, catalogue n° 42017). This medium contains two chromogenic substrates: one for the detection of beta-D-glucuronidase (Escherichia coli) producing pink colonies, and the other for the detection of galactosidase (other Enterobacteriaceae than Escherichia coli) producing blue colonies. Both plates were incubated aerobically at 37° C. during 24 h.

Enterococcus spp were evaluated on Enterococci agar (Merck, Darmstadt, Germany, catalogue n° 65009) and Staphylococcus spp on Baird Parker agar (AES Cheminex, Combourg, France, catalogue n° AEB150302) after aerobic incubation at 37° C. during 48 h. After heating the sample in a water bath at 80° C. during 10 min, Clostridium perfringens was isolated using TSN agar (BioMérieux, Marcy l'Etoile, France, catalogue n° 51048) incubated in an anaerobic chamber at 46° C. during 24 hours.

Identities of representative colonies were furthermore confirmed by microscopic examination after gram staining and biochemical testing using the appropriate API system (BioMérieux, Marcy l'Etoile, France).

The respective bacterial counts (number of colony forming units (CFU) per gram of ileal content dry matter (DM)) are presented in FIG. 8 (FIG. 8: Ileo-rectal microflora in ten piglets submitted to an ileo-rectal anastomosis fed a diet without or with supplementation of 150 ppm aureusidin), which also shows the amendment in the respective bacterial counts caused by addition of 150 ppm of aureusidin, relative to the control.

In this assay, no significant differences in the bacterial counts were found, only numeric effects were observed, but some times they were very strong.

A neutral effect of aureusidin was seen on the mean of total lactic acid bacteria where the counts were decreased by 17% relative to the control. But a positive effect of aureusidin was also seen on the mean of Enterobacteriacae and Escherichia coli where the counts were respectively decreased by 30% and 66% relative to the control.

### EXAMPLE 12

### Evaluation of Compounds in the Weaner Piglet

### Summary of the Experimental Design

Animals: 114 weaner piglets (7.7±0.76 kg—28 days old), divided into six equal groups (A, B, C, D, E and F).

Diets:

| Ingredients (%) | A | B | C | D | E | F |
|---|---|---|---|---|---|---|
| Soybean meal | 7.4 | | | | | |
| Wheat | 15 | | | | | |
| Barley | 29.4 | | | | | |
| Potato concentrate | 8 | | | | | |
| Maize | 10 | | | | | |
| Wheat bran | 1.6 | | | | | |
| Oatmeal | 10 | | | | | |
| Beet pulp | 5.5 | | | | | |
| Treacle | 3.5 | | | | | |
| Soya oil | 2.7 | | | | | |
| Minerals, vitamins, | 6.9 | | | | | |
| synthetic amino acids | | | | | | |
| Mixture 1 (ppm) | — | 150 | — | — | — | — |
| 4-cinnamylphenol (ppm) | — | — | 400 | — | — | — |
| Dihydroeugenol (ppm) | — | — | — | 400 | — | — |
| 2,6-dimethylphenol (ppm) | — | — | — | — | 400 | — |
| Aureusidin (ppm) | — | — | — | — | — | 400 |

Calculated content: digestible energy - 13.9 MJ/kg, crude protein - 17.7%, crude fat - 4.9%; amino acids (%) - lysine - 0.95, methionine + cystine - 0.75, threonine - 0.82, arginine - 1.04, tryptophane - 0.21; minerals (%) - phosphorus - 0.60, calcium - 0.75, sodium - 0.23, potassium - 0.70, magnesium - 0.12, chloride - 0.45.

Experimental conditions: The feed has been distributed ad libitum in a mash form under controlled consumption determination and performance calculated (daily weight gain, feed intake and feed conversion ratio).

Observation period: 29 days.

Measurements: Daily weight gain, feed intake and feed conversion ratio calculated on days 14th and at the end of the experimental period.

### Experimental Results

Effects of the addition to the diet of plant compounds on the daily weight gain (DWG), the feed intake, the feed conversion ratio (FCR) and the mortality of the weaner piglet. A—basal diet, B—A+150 ppm of mixture 1, C—A+400 ppm of 4-cinnamylphenol, D—A+400 ppm of dihydroeugenol, E—A+400 ppm of 2,6-dimethylphenol and F—A+400 ppm of aureusidin.

| Variables | Day | A | B | C | D | E | F |
|---|---|---|---|---|---|---|---|
| DWG (g) | 0-14 | 181 ± 48(1) | 202 ± 56 | 193 ± 41 | 208 ± 39 | 186 ± 45 | 189 ± 61 |
| | | (100) | (112) | (107) | (115) | (103) | (104) |
| | 0-29 | 260 ± 58(1) | 279 ± 64 | 262 ± 53 | 285 ± 33 | 272 ± 53 | 275 ± 48 |
| | | (100) | (107) | (101) | (110) | (105) | (106) |
| Feed | 0-14 | 339 ± 48(2) | 358 ± 27 | 345 ± 42 | 364 ± 46 | 350 ± 5 | 332 ± 52 |
| intake | | (100) | (106) | (102) | (107) | (103) | (98) |
| (g/day) | 0-29 | 657 ± 70(2) | 688 ± 42 | 670 ± 72 | 708 ± 85 | 694 ± 27 | 686 ± 72 |
| | | (100) | (105) | (102) | (108) | (106) | (104) |
| FCR | 0-14 | 1.9(2) ± 0.051 | 1.876 ± 0.104 | 1.82 ± 0.058 | 1.776 ± 0.034 | 1.886 ± 0.06 | 1.965 ± 0.21 |
| (kg/kg) | | (100) | (99) | (96) | (93) | (99) | (103) |
| | 0-29 | 1.913(2) ± 0.083 | 1.92 ± 0.086 | 1.953 ± 0.048 | 1.88 ± 0.08 | 1.914 ± 0.053 | 1.968 ± 0.018 |
| | | (100) | (100) | (102) | (98) | (100) | (103) |
| Mortality | 0-29 | — | — | — | — | — | — |

Diet based on: wheat, maize, barley and soybean meal;
Animals: piglets of an initial body weight of 7.7 ± 0.76 kg;
(1)Mean ± standard deviation of the mean of 19 determinations;
(2)Mean ± standard deviation of the mean of 3 determinations.

Conclusion: Mixture 1 and dihydroeugenol improved the daily weight gain of the piglets, comparatively to the non supplemented control for the whole observation period, by 7 and 10% respectively. Furthermore, the animals ingesting dihydroeugenol had a better feed conversion ratio than the controls. The improvement was of 7% for the first 14 days of the experiment and of 2% for the whole observation period.

1.-11. (canceled)

12. Use of a compound as defined by formula (I) 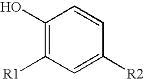
wherein
R1 is hydrogen or hydroxyl or OR3, wherein the residues R3 is a lower alkyl or lower alkenyl residue;
R2 is hydrogen or a lower alkyl or lower alkoxy or lower alkenyl residue as a component of animal feed or feed additives for improving the performance of animals.

13. Use of a compound of formula (I), wherein the compound is dihydroeugenol (2-methoxy-4-propylphenol).

14. Use of a compound according to claim 12 for the preparation of compositions for improving the performance of animals, especially having an activity as modulators of the gastrointestinal microflora of animals and which are applicable via animal feed.

15. Feed or food additive comprising as active ingredient a compound as defined by formula (I) 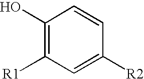
wherein
R1 is hydrogen or hydroxyl or OR3, wherein the residues R3 is a lower alkyl or lower alkenyl residue;
R2 is hydrogen or a lower alkyl or lower alkoxy or lower alkenyl residue.

16. Feed or food additive according to claim 12 comprising as active ingredient dihydroeugenol (2-methoxy-4-propylphenol).

17. An animal feed additive according to claim 15 comprising
(a) at least one fat-soluble vitamin,
(b) at least one water-soluble vitamin,
(c) at least one trace mineral, and/or
(d) at least one macro mineral.

18. Nutritional composition comprising as active ingredient a compound as defined by formula (I) 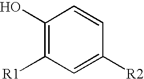
wherein
R1 is hydrogen or hydroxyl or OR3, wherein the residues R3 is a lower alkyl or lower alkenyl residue;
R2 is hydrogen or a lower alkyl or lower alkoxy or lower alkenyl residue.

19. Animal feed according to claim 12, which improves animal feed utilization by improving the feed conversion ratio (FCR), and/or modulating the gut microflora.

20. An animal feed composition according to claim 19 having a crude protein content of 50 to 800 g/kg feed.

21. A method of feeding an animal with a feedstuff, wherein a compound as defined by formula (I) 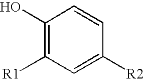
is added to the feed, and wherein
R1 is hydrogen or hydroxyl or OR3, wherein the residues R3 is a lower alkyl or lower alkenyl residue;
R2 is hydrogen or a lower alkyl or lower alkoxy or lower alkenyl residue.

22. A method according to claim 21 for improving animal feed conversion ratio (FCR) and/or modulating animal gut microflora.
